SEQUENCE LISTING (56 sequences)

SEQ ID NO: 1 - protein, 324 aa, Homo sapiens
FLYQVSTHEQSESSHGRSGTSTGGRQGSHHEQARDSSRHSTSQEGQDTIHGHPGSSSGGRQGSHYEQSVDRSGHSGSHHSHTTSQGRSDASHGTSGSRSASRQTRNQEQSGDGSRHSGSRHHEASSRADSSRHSQVGQGESSGPRTSRNQGSSFSQDSDSQGHSEDSERWSGSASRNHHGSAQEQSRDGSRHPRSHQEDRAGHGHSADSSRQSGTRHTQTSSGGQAASSHEQARSSAGDRHGSGHQQSADSSRHSGIGHGQASSAVRDSGHRGSSGSQASDNEGHSEDSDTQSVSAHGQAGSHQQSHQESTRGRSRGRSGRSGS

SEQ ID NO: 2 - protein, 13 aa, Artificial Sequence
  A synthetic peptide
QELKLXSXTKSSV

SEQ ID NO: 3 - protein, 15 aa, Artificial Sequence
  A synthetic peptide
CQELKLXSXTKSSVC

SEQ ID NO: 4 - protein, 13 aa, Artificial Sequence
  A synthetic peptide
EHYHYXAXGHTTT

SEQ ID NO: 5 - protein, 15 aa, Artificial Sequence
  A synthetic peptide
CEHYHYXAXGHTTTC

SEQ ID NO: 6 - protein, 13 aa, Artificial Sequence
  A synthetic peptide
QQFRFXGXSRAAC

SEQ ID NO: 7 - protein, 14 aa, Artificial Sequence
  A synthetic peptide
CQQFRFXGXSRAAC

SEQ ID NO: 8 - protein, 13 aa, Artificial Sequence
  A synthetic peptide
HEFRFXGXSRAAC

SEQ ID NO: 9 - protein, 14 aa, Artificial Sequence
  A synthetic peptide
CHEFRFXGXSRAAC

SEQ ID NO: 10 - protein, 13 aa, Artificial Sequence
  A synthetic peptide
HHFRFXGXSRAAC

SEQ ID NO: 11 - protein, 14 aa, Artificial Sequence
  A synthetic peptide
CHHFRFXGXSRAAC

SEQ ID NO: 12 - protein, 13 aa, Artificial Sequence
  A synthetic peptide
HQLRFXGXSRAAC

SEQ ID NO: 13 - protein, 14 aa, Artificial Sequence
  A synthetic peptide
CHQLRFXGXSRAAC

SEQ ID NO: 14 - protein, 13 aa, Artificial Sequence
  A synthetic peptide
HQFKFXGXSRAAC

SEQ ID NO: 15 - protein, 14 aa, Artificial Sequence
  A synthetic peptide
CHQFKFXGXSRAAC

SEQ ID NO: 16 - protein, 13 aa, Artificial Sequence
  A synthetic peptide
HQFRLXGXSRAAC

SEQ ID NO: 17 - protein, 14 aa, Artificial Sequence
  A synthetic peptide
CHQFRLXGXSRAAC

SEQ ID NO: 18 - protein, 13 aa, Artificial Sequence
  A synthetic peptide
HQFRFXSXSRAAC

SEQ ID NO: 19 - protein, 14 aa, Artificial Sequence
  A synthetic peptide
CHQFRFXSXSRAAC

SEQ ID NO: 20 - protein, 13 aa, Artificial Sequence
  A synthetic peptide
HQFRFXGXSKAAC

SEQ ID NO: 21 - protein, 14 aa, Artificial Sequence
  A synthetic peptide
CHQFRFXGXSKAAC

SEQ ID NO: 22 - protein, 13 aa, Artificial Sequence
  A synthetic peptide
HQFKFXGXSKAAC

SEQ ID NO: 23 - protein, 14 aa, Artificial Sequence
  A synthetic peptide
CHQFKFXGXSKAAC

SEQ ID NO: 24 - protein, 13 aa, Artificial Sequence
  A synthetic peptide
HQFRFXGXTRAAC

SEQ ID NO: 25 - protein, 14 aa, Artificial Sequence
  A synthetic peptide
CHQFRFXGXTRAAC

SEQ ID NO: 26 - protein, 13 aa, Artificial Sequence
  A synthetic peptide
HQFRFXGXSRSAC

SEQ ID NO: 27 - protein, 14 aa, Artificial Sequence
  A synthetic peptide
CHQFRFXGXSRSAC

SEQ ID NO: 28 - protein, 13 aa, Artificial Sequence
  A synthetic peptide
HQFRFXGXSRASC

SEQ ID NO: 29 - protein, 14 aa, Artificial Sequence
  A synthetic peptide
CHQFRFXGXSRASC

SEQ ID NO: 30 - protein, 13 aa, Artificial Sequence
  A synthetic peptide
HQSRFXGXSRAAC

SEQ ID NO: 31 - protein, 14 aa, Artificial Sequence
  A synthetic peptide
CHQSRFXGXSRAAC

SEQ ID NO: 32 - protein, 13 aa, Artificial Sequence
  A synthetic peptide
HQFRFXAXSRAAC

SEQ ID NO: 33 - protein, 14 aa, Artificial Sequence
  A synthetic peptide
CHQFRFXAXSRAAC

SEQ ID NO: 34 - protein, 13 aa, Artificial Sequence
  A synthetic peptide
HQFRFXGXGRAAC

SEQ ID NO: 35 - protein, 14 aa, Artificial Sequence
  A synthetic peptide
CHQFRFXGXGRAAC

SEQ ID NO: 36 - protein, 19 aa, Artificial Sequence
  A synthetic peptide
SHQESTRGRSRGRSGXSGS

SEQ ID NO: 37 - protein, 19 aa, Artificial Sequence
  A synthetic peptide
SHQESTXGXSRGRSGRSGS

SEQ ID NO: 38 - protein, 19 aa, Artificial Sequence
  A synthetic peptide
SHQESTXGRSXGRSGRSGS

SEQ ID NO: 39 - protein, 19 aa, Artificial Sequence
  A synthetic peptide
SHQESTXGRSRGRSGXSGS

SEQ ID NO: 40 - protein, 19 aa, Artificial Sequence
  A synthetic peptide
SHQESTXGRSRGXSGRSGS

SEQ ID NO: 41 - protein, 19 aa, Artificial Sequence
  A synthetic peptide
SHQESTXGRSRGRSGRSGS

SEQ ID NO: 42 - protein, 19 aa, Artificial Sequence
  A synthetic peptide
SHQESTRGRSXGRSGRSGS

SEQ ID NO: 43 - protein, 19 aa, Artificial Sequence
  A synthetic peptide
SHQESTRGRSRGXSGRSGS

SEQ ID NO: 44 - protein, 19 aa, Artificial Sequence
  A synthetic peptide
SHQESTRGXSRGRSGRSGS

SEQ ID NO: 45 - protein, 19 aa, Artificial Sequence
  A synthetic peptide
SHQESTXGRSRGRSGRSGS

SEQ ID NO: 46 - protein, 14 aa, Artificial Sequence
  A synthetic peptide
SHQESTXGRSRGRS

SEQ ID NO: 47 - protein, 13 aa, Artificial Sequence
  A synthetic peptide
SHQESTXGRSRGR

SEQ ID NO: 48 - protein, 11 aa, Artificial Sequence
  A synthetic peptide
SHQESTXGRSR

SEQ ID NO: 49 - protein, 12 aa, Artificial Sequence
  A synthetic peptide
SHQESTXGRSRG

SEQ ID NO: 50 - protein, 10 aa, Artificial Sequence
  A synthetic peptide
SHQESTXGRS

SEQ ID NO: 51 - protein, 9 aa, Artificial Sequence
  A synthetic peptide
SHQESTXGR

SEQ ID NO: 52 - protein, 19 aa, Artificial Sequence
  A synthetic peptide
SHQESTRGRSRGRSGXSGS

SEQ ID NO: 53 - protein, 19 aa, Artificial Sequence
  A synthetic peptide
SHQESTXGXSRGRSGRSGS

SEQ ID NO: 54 - protein, 19 aa, Artificial Sequence
  A synthetic peptide
SHQESTXGRSXGRSGRSGS

SEQ ID NO: 55 - protein, 19 aa, Artificial Sequence
  A synthetic peptide
SHQESTXGRSRGRSGXSGS

SEQ ID NO: 56 - protein, 19 aa, Artificial Sequence
  A synthetic peptide
SHQESTXGRSRGXSGRSGS

(19) United States
(12) Patent Application Publication
(10) Pub. No.: US 2011/0014632 A1
(43) Pub. Date: Jan. 20, 2011

(54) ASSAYS FOR CLINICAL ASSESSMENTS OF RHEUMATOID ARTHRITIS

(75) Inventors: Thomas Holzman, Libertyville, IL (US); Winton Gibbons, Winnetka, IL (US); Claude Lerner, Highland Park, IL (US)

Correspondence Address: GREGORY T. PLETTA;Nanosphere, Inc., 4088 Commerical Avenue, Northbrook, IL, 60062-1829, US

(73) Assignee: NANOSPHERE, INC., Northbrook, IL (US)

(21) Appl. No.: 12/537,182

(22) Filed: Aug. 6, 2009

Related U.S. Application Data
(63) Continuation of application No. 12/330,494, filed on Dec. 8, 2008, now abandoned.

(51) Int. Cl. G01N 33/53; C07K 7/08; C07K 7/06; C07K 7/64; G01N 33/564

(52) U.S. Cl. 435/7.9; 530/327; 530/326; 530/328; 530/317; 436/507; 435/7.1; 977/920

(57) ABSTRACT

The disclosure provides methods of detecting autoantibodies present in the serum of subjects suffering from rheumatoid arthritis. The methods use capture probes and detection probes that can bind to the antifilaggrin autoantibodies or other epitope related autoantibodies. The presence, absence, and/or amount of the autoantibody complex may be detected, wherein the presence of the complex may indicate a positive diagnosis of rheumatoid arthritis.

### REFERENCE TO RELATED APPLICATIONS

This application is a continuation of U.S. application Ser. No. 12/330,494, filed Dec. 8, 2008, the complete disclosure of which is incorporated herein by reference.

### TECHNICAL FIELD

The present technology relates generally to diagnostic and prognostic methods for human disease. In particular, the present disclosure relates to methods for detecting autoantibodies (AAs) which are a marker for autoimmune disease, including rheumatoid arthritis (RA).

### BACKGROUND

The following description is provided to assist the understanding of the reader. None of the information provided or references cited is admitted to be prior art to the present invention.

Rheumatoid arthritis (RA) is an auto-inflammatory disease that causes pain, swelling, stiffness, and loss of joint function. RA affects at least one percent (1%) of the U.S. population (˜2.5 million individuals). Currently, the cause of RA is unknown and while there is no cure, a certain level of control of RA can be achieved through the use of biologic drugs, physical exercise, joint protection techniques and self-management methods. Early diagnosis of RA and early, aggressive treatment can help prevent joint damage, deformity and loss of physical mobility. Confirmations of diagnoses are achieved through a combination of diagnostic tests, physical examination, x-ray evidence and imaging approaches.

Diseased cells are often characterized by the production of disease-associated marker proteins. Freedland et al., Jama, 294:433-439 (2005). These consist of aberrant forms of wild-type proteins, which are produced by disease cells as a result of genetic mutations, alternative haplotypes, or altered post-transcriptional or post-translational processing. Soussi T., Oncogene, 26:2145-2156 (2007). Alternatively, disease markers can also be proteins that become over-expressed in diseased cells, usually as a result of gene amplification or abnormal transcriptional regulation. In some cases, these two phenomena may occur at the same time leading to an accumulation of modified proteins throughout the development of the disease. For example, variant forms of filaggrin protein are associated with rheumatoid arthritis (RA). Expression of the disease-associated marker proteins may result in production of AAs against these self-antigens. The AAs may be detected in serum and are therefore useful as diagnostic markers for disease.

### SUMMARY

The technology disclosed herein relates to the detection of AAs for the diagnosis of disease, and a unique set of peptides that bind autoantibodies in the serum of subjects suffering from RA. In particular, the present disclosure provides a method for the diagnosis of RA in a subject comprising: (a) providing a substrate having a capture probe bound thereto, wherein the capture probe comprises an antigen recognized by autoantibodies present in the serum of subjects suffering from rheumatoid arthritis; (b) contacting the substrate having the capture probe bound thereto with (i) a sample from the subject and (ii) a detection probe under conditions that are suitable for the formation of a complex of the capture probe and detection probe with the autoantibodies, if present in the sample, wherein the detection probe comprises a nanoparticle and a binding agent that specifically binds to the autoantibodies; and (c) detecting the formation of the complex of the capture probe and detection probe with the autoantibodies, wherein the presence of the complex is indicative of rheumatoid arthritis in the subject.

In one embodiment, the antigen recognized by autoantibodies present in the serum of subjects suffering from RA is a citrullinated polypeptide or a fragment thereof. In one embodiment, the antigen recognized by autoantibodies present in the serum of subjects suffering from RA is a citrullinated peptide. In a particular embodiment, the citrullinated peptide is a cyclic citrullinated peptide. In suitable embodiments, the citrullinated peptide has a sequence according to any of SEQ ID NOs: 2-55.

In one embodiment, the sample is first contacted with the detection probe and then contacted with the capture probe. In another embodiment, the sample is first contacted with the capture probe and then contacted with the detection probe. In yet another embodiment, the sample, the detection probe, and the capture probe are contacted simultaneously.

In one embodiment the binding agent that specifically binds to the autoantibodies is an anti-human Ig antibody. For example, the anti-human antibody is selected from the group consisting of: anti-human IgG, anti-human IgM, anti-human IgA, anti-human IgE, anti-human IgD, and subtypes or mixtures thereof. In one embodiment, the detection probe further comprises a fluorophore, a phosphor, a quantum dot, an enzyme conjugate, or an avidin/biotin conjugate.

In one embodiment, the nanoparticle is conjugated directly to the binding agent. In another embodiment, the nanoparticle is conjugated indirectly to the binding agent by a bridge or linker molecule. For example, the nanoparticle and binding agent may each be conjugated to biotin and the nanoparticle and second binding agent may be joined by an avidin or streptavidin bridge.

In one embodiment, the complex is detected by photonic, electronic, acoustic, opto-acoustic, gravitic, electro-chemical, electro-optic, mass-spectrometric, enzymatic, chemical, biochemical, magnetic, paramagnetic, or physical means. In one embodiment, the detecting step comprises contacting the substrate with silver stain. In one embodiment, the detecting comprises detecting light scattered by the nanoparticles.

In one embodiment, the nanoparticles are made of a noble metal, e.g., gold or silver. In one embodiment, the substrate is a nanoparticle, a thin film, or a magnetic bead. In one embodiment, the substrate has a planar surface and is made of glass, quartz, ceramic, or plastic. In some embodiments, the substrate is addressable.

In one embodiment, the subject is a human. In a suitable embodiment, the sample is blood, plasma, or serum.

### DETAILED DESCRIPTION

Disease-associated marker proteins may be found both in the tissues and in the bodily fluids of an individual who suffers from a disease or medical condition. Their levels are very low at the early stages of the disease process and increase during progression of the disease. AAs produced by patients suffering from certain diseases specifically recognize disease-associated marker proteins. The detection of AAs produced by patients with disease may therefore be used to design alternative, more reliable and sensitive tests to detect the disease condition in an individual from the very beginning of their occurrence. In particular, AAs that recognize citrullinated antigens, e.g., filaggrin, filaggrin variants, or CCP, have been identified in patients suffering from rheumatoid arthritis.

In the description that follows, a number of terms are utilized extensively. Definitions are herein provided to facilitate understanding of the invention. The terms described below are more fully defined by reference to the specification as a whole. In practicing the invention, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y. (1989)); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press (1986)); Perbal, A Practical Guide to Molecular Cloning; the series, Meth. Enzymol., (Academic Press, Inc. (1984)); Gene Transfer Vectors for Mammalian Cells, Miller & Calos, Eds. (Cold Spring Harbor Laboratory, NY (1987); and Meth. Enzymol., Vols. 154 and 155, Wu & Grossman, and Wu, Eds., respectively. Units, prefixes, and symbols may be denoted in their accepted SI form.

Unless defined otherwise, all technical and scientific terms used herein generally have the same meaning as commonly understood by one of ordinary skill in the art to which this invention belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well known and commonly employed in the art.

As used herein, the term “array” refers to a population of different molecules (e.g., capture probes) that are attached to one or more substrates such that the different probe molecules can be differentiated from each other according to relative location. An array can include different probe molecules that are each located at a different addressable location on a substrate. Alternatively, an array can include separate substrates each bearing a different probe molecule. Probes attached to separate substrates can be identified according to the locations of the substrates on a surface to which the substrates are associated or according to the locations of the substrates in a liquid. As used herein, the term “addressable array” or “addressable substrate” refers to an array wherein the individual elements have precisely defined coordinates, so that a given element at a particular position in the array can be identified.

The term “antigen” refers to is a substance that prompts the generation of antibodies and can cause an immune response. Examples of antigens include, but are not limited to, citrullinated filaggrin or filaggrin variants or fragments, citrullinated peptides, and cyclic citrullinated peptides (CCP) that are immunologically reactive or cross-reactive with AAs present in the serum of RA patients.

As used herein, the term “antibody” means a polypeptide comprising a framework region from an immunoglobulin gene or fragments thereof that specifically binds and recognizes an antigen. Use of the term antibody is meant to include whole antibodies, including single-chain whole antibodies, antibody fragments such as Fab fragments, and other antigen-binding fragments thereof. The term “antibody” includes bispecific antibodies and multispecific antibodies so long as they exhibit the desired biological activity or function.

An “autoantibody” (abbreviated “AA”) is an antibody produced by the immune system of a subject that is directed against one or more of the subject's own proteins.

As used herein, the term “binding agent” is a compound, a macromolecule, including polypeptide, DNA, RNA and carbohydrate that selectively binds a target molecule. For example, a binding agent can be a polypeptide that selectively binds with high affinity or avidity to a target analyte without substantial cross-reactivity with other polypeptides that are unrelated to the target analyte. The affinity of a binding agent that selectively binds a target analyte will generally be greater than about 10⁻⁵ M, such as greater than about 10⁻⁶ M, including greater than about 10⁻⁸ M and greater than about 10⁻⁹ M. Specific examples of such selective binding agents include a polyclonal or monoclonal antibody specific for a disease-associated antigen or human immunoglobulin. The binding agent can be labeled with a detectable moiety, if desired, or rendered detectable by specific binding to a detectable secondary binding agent.

As used herein, the term “capture probe” refers to a molecule capable of binding to a target analyte, e.g., a disease-associated AA. One example of a capture probe includes antigens that recognize AAs present in a biological sample from patients having or suspected of having a disease, e.g., RA. Other examples of capture probes include aptamers, protein ligands, etc., which are described for instance, in PCT/US01/10071 (Nanosphere, Inc.).

As used herein, the term “complex” means an aggregate of two or more molecules that result from specific binding between the molecules, such as an antibody and an antigen, a receptor and a ligand, etc.

A “detection probe” is a labeled molecule including one or more binding agents, wherein the one or more binding agents specifically bind to a specific target analyte. The label itself may serve as a carrier, or the probe may be modified to include a carrier. Carriers that are suitable for the methods include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

As used herein, the term “disease-associated antigen,” refers to a substance associated with a disease or medical condition in a subject, e.g., RA, which causes an autoimmune response in that subject, resulting in the production of AAs. Disease-associated antigens include the wild-type protein, complexes, and aggregates as well as modified forms (mutants, haplotypes, or other variant forms), complexes, and aggregates of wild-type proteins.

As used herein, the term “epitope related antibody” (abbreviated “ERA”) includes immunologically cross-reactive antibodies to homologs, metabolites, and variants of antigens associated with RA. ERAs may recognize functionally equivalent antigens seen in, e.g., (1) non human primates, rodents, canines, and other animal models; (2) derived tissue models, as well as (3) native or genetically engineered or assembled cellular assay models.

The term “haplotype” as used herein is intended to refer to a set of alleles that are inherited together as a group (are in linkage disequilibrium) at statistically significant levels (p_{corr}<0.05). In the context of the present invention, a haplotype preferably refers to a combination of biallelic marker alleles found in a given individual and which may be associated with a phenotype.

The term “homology” refers to sequence similarity between two peptides or between two nucleic acid molecules. Homology may be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences.

“Identity” means the degree of sequence relatedness between polypeptide or polynucleotide sequences, as the case may be, as determined by the match between strings of such sequences. “Identity” and “homology” can be readily calculated by known methods. Suitable computer program methods to determine identity and homology between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990).

As used herein, the terms “immunologically cross-reactive” and “immunologically-reactive” are used interchangeably to mean an antigen which is specifically reactive with an antibody which was generated using the same (“immunologically-reactive”) or different (“immunologically cross-reactive”) antigen.

As used herein, the term “immunologically-reactive conditions” means conditions which allow an antibody to bind to that epitope or a structurally similar epitope to a detectably greater degree than the antibody binds to substantially all other epitopes, generally at least two times above background binding, preferably at least five times above background. Immunologically-reactive conditions are dependent upon the format of the antibody binding reaction and typically are those utilized in immunoassay protocols. See, Harlow & Lane, Antibodies, A Laboratory Manual (Cold Spring Harbor Publications, New York (1988), for a description of immunoassay formats and conditions.

As used herein, the terms “label” or “detectable label” refers to a marker that may be detected by photonic, electronic, opto-electronic, magnetic, gravitic, acoustic, enzymatic, magnetic, paramagnetic, or other physical or chemical means. The term “labeled” refers to incorporation of such a detectable marker, e.g., by incorporation of a radiolabeled molecule or attachment to a nanoparticle.

As used herein, the term “level” is intended to mean the amount, accumulation or rate of synthesis of a molecule. The term can be used to refer to an absolute amount of a molecule in a sample or to a relative amount of the molecule, including amounts determined under steady-state or non-steady-state conditions. The level of a molecule can be determined relative to a control molecule in a sample. The level of a molecule also can be referred to as an expression level.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example.

The term “ortholog” refers to genes or proteins which are homologs via speciation, e.g., closely related and assumed to have common descent based on structural and functional considerations. Orthologous proteins function as recognizably the same activity in different species. The term “paralog” denotes a polypeptide or protein obtained from a given species that has homology to a distinct polypeptide or protein from that same species.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “reference level” is intended to mean a control level of a biomarker, e.g., disease-associated AA, used to evaluate a test level of the biomarker in a sample from an individual. A reference level can be a normal reference level or a disease-state reference level. A normal reference level is an amount of expression of a biomarker in a non-diseased subject or subjects. A disease-state reference level is an amount of expression of a biomarker in a subject with a positive diagnosis for the disease or condition. A reference level also can be a stage-specific reference level. A stage-specific reference level refers to a level of a biomarker characteristic of a given stage of progression of a disease or condition.

As used herein, the term “sample” means sample material derived from or contacted by living cells. The term “sample” is intended to include tissues, cells and biological fluids isolated from a subject, as well as tissues, cells and fluids present within a subject. Biological samples include, e.g., but are not limited to, whole blood, plasma, serum, semen, cell lysates, saliva, tears, urine, fecal material, sweat, buccal, skin, cerebrospinal fluid, and hair. Biological samples can also be obtained from biopsies of internal organs. Biological samples can be obtained from subjects for diagnosis or research or can be obtained from undiseased individuals, as controls or for basic research.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

As used herein the phrase “splice variant” refers to mRNA molecules produced from primary RNA transcripts that have undergone alternative RNA splicing. Alternative RNA splicing occurs when a primary RNA transcript undergoes splicing, generally for the removal of introns, which results in the production of more than one mRNA molecule each of which may encode different amino acid sequences. The term “splice variant” also refers to the proteins encoded by the above mRNA molecules.

As used herein, the term “subject” means the subject is a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substitution” is one of mutations that is generally used in the art. Substitution variants have at least one amino acid residue in a polypeptide molecule replaced by a different residue. “Conservative substitutions” typically provide similar biological activity as the unmodified polypeptide sequence from which the conservatively modified variant was derived. Conservative substitutions typically include the substitution of one amino acid for another with similar characteristics. Conservative substitution tables providing functionally similar amino acids are well known in the art. For example, the following six groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic (Cationic): Arginine (R), Lysine (K), Histidine (H); Acidic (Anionic): Aspartic acid (D), Glutamic acid (E); Amide: Asparagine (N), Glutamine (Q).

As used herein, the term “substrate” refers to any surface capable of having capture probes bound thereto. Such surfaces include, but are not limited to, glass, metal, plastic, or materials coated with a functional group designed for binding of capture probes or analytes. Substrates also may be referred to as slides.

As used herein, the terms “treating,” “treatment,” or “alleviation” refers to both therapeutic treatment and prophylactic or preventative measures, wherein the object is to prevent or slow down (lessen) the targeted pathologic condition or disorder. A subject is successfully “treated” for a disorder characterized by increased autoantibody levels if the subject shows observable and/or measurable reduction in or absence of one or more signs and symptoms of a particular disease or condition.

As used herein, the term “variant polypeptide” refers to a polypeptide that differs from a naturally occurring polypeptide in amino acid sequence or in ways that do not involve amino acid sequence modifications, or both. Non-sequence modifications include, but are not limited to, changes in citrullination, acetylation, methylation, phosphorylation, carboxylation, or glycosylation. Variants may also include sequences that differ from the wild-type sequence by one or more amino acid substitutions, deletions, or insertions. The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

### Diagnostic Methods for RA

The development of immunologic responsiveness to self is called autoimmunity and reflects the impairment of self-tolerance. Immunologic, environmental, and genetic factors are closely interrelated in the pathogenesis of autoimmunity. The frequency of autoimmune antibodies (AAs) in the general population increases with age, suggesting a breakdown of self-tolerance with aging. AAs also may develop as an aftermath of disease tissue damage.

The development of autoimmunity usually involves the breakdown or circumvention of self-tolerance. The potential for the development of AAs probably exists in most individuals. For example, normal human B cells are capable of reacting with several self-antigens, but are suppressed from producing AAs by one or more tolerance mechanisms. Precommitted B cells in tolerant individuals can be stimulated in several ways. For example, tolerance involving only T cells, induced by persistent low levels of circulating self-antigens, may breakdown in the presence of substances such as endotoxin. Such substances stimulate the B cells directly to produce AAs. Another tolerance mechanism involves suppressor T cells. A decrease in suppressor T cell activity therefore may also lead to production of AAs.

In various embodiments, the methods described herein may be used to detect AAs raised against antigens associated with RA. A disease-associated antigen may be a variant form of a polypeptide, i.e., a polypeptide formed as the result of mutation or alternative post-transitional modification. Such variants are also referred to herein as “neopeptides.” A number of antigens associated with RA have been described in the literature. See Blass et al., The Immunologic Homunculus in Rheumatoid Arthritis. Arthritis and Rheumatism 1999; 42:2499-2506. Some antigens associated with RA are well characterized biochemically and by their antigenic character. For example, the Sa and filaggrin antigens are antigens that are not present in the inflamed joint as such, but draw attention as targets of very disease-specific immune responses. The Sa antigen is a 50 k protein isolated from human spleen or placenta. Sa-specific antibodies occur in RA patients with a 43% sensitivity and a 78% to 99% specificity. Filaggrin is a 42 k protein involved in the crosslinking of intermediate filament proteins, namely, cytokeratin, and is present in the endothelium. Antibodies to filaggrin seem to be identical to previously described antiperinuclear factor and antikeratin antibodies. The major determinant of the epitope(s) targeted by antifilaggrin antibodies is citrulline, a modified arginine residue. Specific examples of AAs that have been associated with RA include, but are not limited to rheumatoid factors (RFs), antibodies to citrullinated antigens such as filaggrin and anti-CCP antibody, and antibodies to immunoglobulin binding protein (BiP).

To date, strategies for detecting anti-filaggrin antibodies (AFAs) have focused on a series of peptides that are natural mimics of the filaggrin amino acid sequence (refer to U.S. Pat. No. 7,335,724 B2 and references therein; also see Table 1, SEQ ID NO. 1). These peptides, and cyclized variants of these peptides, have shown high specificity and sensitivity, but none of the peptides designed to date have reached >80% sensitivity with a high specificity See Bizarro et al., Clinical Chemistry, 53:1527-1533 (2007). By contrast, the peptides disclosed herein enable a more specific and sensitive assay for RA to be developed. In one embodiment, rationally designed artificial polypeptide sequences can be used to detect the presence, absence, and/or amount of anti-filaggrin antibodies (AFA) in a biological sample (see Table 2). Elevated AFAs can be found in patients who have a negative RF, the classic test for RA. In some embodiments, the AFAs may specifically bind one or more of the artificial polypeptide sequences, including citrullinated forms of the polypeptide or polypeptides. The filaggrin gene (FLG) is located within the epidermal differentiation complex (EDC) on 1q21.3, a gene cluster expressed late in epidermal differentiation. FLG contains a large and highly repetitive exon 3, which also shows population size variation (12.7-14.7 kb). This exon encodes 10-12 full tandem repeats of the filaggrin protein that are almost 100% identical at the DNA sequence level, flanked by two partial repeats. A number of filaggrin variants have been identified as associated with certain diseases. For example, three loss-of-function variants of the filaggrin gene have been discovered: R501X, 2282del4 and 3702del1. In some RA patients, heterozygous carriers of either of the these FLG variants exhibited a significantly elevated prevalence of AAs to citrullinated peptides (CCP-2) (80%) compared to non-carriers (51.9%) (Huffineier et al., Ann Rheum Dis 2008; 67:131-133.

In one embodiment, the detection of AAs in a sample is indicative of atopic dermatitis or ichthyosis vulgaris in the subject. A strong association between the occurrence of atopic dermatitis or ichthyosis vulgaris and 15 filaggrin variants was established (Sandilands et al., Nature Genetics 2007; 39: 650-654). These variants include: R501X, 2282del4, 2702delG, R1474X, 5360delG, 6687delAG, E2422X, 7267delCA, R2477X, S3247X, 11029delCA, 11033del4, Q3683X, 3321delA, and S2554X. Some of these variants also show a strong association with moderate-to-severe childhood eczema. The data described in these studies suggest that haplotypes of the filaggrin gene consist of both prevalent and rare risk alleles. Other citrullinated polypeptide variants have been associated with RA as well as other autoimmune diseases. For example, Vimentin also has been used as an antigen for detecting AAs associated with RA. Furthermore, citrullination of histones has been implicated in multiple sclerosis (MS) (Matronardi et. al, J. Neuroscience, 2006, 26, 11387). Rational design of artificial peptide sequences such as those described in Table 2 potentially can be used for diagnosis of MS and other autoimmune diseases where citrullination is involved.

In one aspect, the disclosure provides methods of detecting AAs associated with RA in biological samples. In one embodiment, the method comprises contacting a sample with a capture probe comprising an antigen recognized by the target analytes (e.g., AAs) and nanoparticles having anti-human Ig antibodies attached thereto. For example, the capture probe can bind to an AA and the nanoparticle probe comprising a detection antibody can also bind to the AA thereby forming a sandwich complex. The presence, absence, and/or amount of the complex may be detected, wherein the presence or absence of the complex is indicative of the presence, absence, or amount of the AAs. As described above, certain AAs are biomarkers for RA.

In a suitable embodiment, the method comprises using a sandwich assay to detect the AAs. Sandwich assays generally involve the use of binding molecules (e.g. antibodies), each capable of binding to a different immunogenic portion, or epitope, of the protein to be detected and/or quantitated. In a sandwich assay, the analyte is typically bound by a first binding molecule which is immobilized on a solid support, and thereafter a second binding molecule binds to the analyte, thus forming an insoluble three part complex. See, e.g., U.S. Pat. No. 4,376,110. In some embodiments of these methods, the first binding molecule is an antigen recognized by AAs, the analyte is the AA, and the second binding molecule is an anti-human Ig antibody which specifically binds to the AA.

In one embodiment, the sample is first contacted with the detection probe so that an AA present in the sample binds to the binding agent on the detector probe, and the AA bound to the detection probe is then contacted with the substrate having capture probes bound thereto so that the AA binds to the capture probes on the substrate. In another embodiment, the sample is first contacted with the substrate so that AAs present in the sample bind to a capture probe, and the AAs bound to the capture probe are then contacted with the detection probe so that the AAs bind to the binding agent on the detection probe. In another embodiment, the sample, the detection probe and the capture probe on the substrate are contacted simultaneously.

An exemplary method for detecting the presence, absence, and/or amount of AAs in a biological sample involves obtaining a biological sample (e.g., blood, serum or plasma) from a test subject and contacting the biological sample with an antigen recognized by AAs such that the presence of the AAs is detected in the biological sample. The amount of binding is compared with a suitable reference sample or control, which can be the amount of binding in the absence of the AAs, the amount of the binding in the presence of a non-specific immunoglobulin composition, or both.

In some embodiments, the antigens recognized by the AAs, when used in a sandwich assay employing gold-nanoparticle detection with silver enhancement, significantly improves the LOD for AAs by lowering the detectable concentration of the complex formed between the antigen and the captured antibody. Additionally, in some embodiments, the assay employs a mixed set of biotinylated secondary antibody isotypes which allow more favorable detection of the response of human anti-CCP antibodies—particularly a mixture of IgG, IgM, IgE, IgD, and IgA and subtypes thereof may be used as detection antibodies.

A variety of antigens may be used as capture probes according to the methods described herein. An example of an antigen recognized by AAs is all or a fragment of SEQ ID NO: 1 (See Table 1), wherein at least one arginine residue is replaced by citrulline. Additional conservative or non-conservative substitutions may be introduced, so long as the antigen is capable of specifically binding to AAs present in the serum of RA patients. Fragments of SEQ ID NO: 1 may comprise at least 5, at least 10, at least 15, at least 20, at least 25, at least 30, at least 50, or at least 100 contiguous amino acids.

TABLE 1
Sequence of Human Filaggrin (SEQ ID NO: 1)

FLYQVSTHEQSESSHGRSGTSTGGRQGSHHEQARDSSRHSTSQEGQDTIH
GHPGSSSGGRQGSHYEQSVDRSGHSGSHHSHTTSQGRSDASHGTSGSRSA
SRQTRNQEQSGDGSRHSGSRHHEASSRADSSRHSQVGQGESSGPRTSRNQ
GSSFSQDSDSQGHSEDSERWSGSASRNHHGSAQEQSRDGSRHPRSHQEDR
AGHGHSADSSRQSGTRHTQTSSGGQAASSHEQARSSAGDRHGSGHQQSAD
SSRHSGIGHGQASSAVRDSGHRGSSGSQASDNEGHSEDSDTQSVSAHGQA
GSHQQSHQESTRGRSRGRSGRSGS

In some embodiments, the antigen is a citrullinated peptide that is immunologically cross-reactive with AAs present in the serum of subjects suffering from rheumatoid arthritis. For example, the citrullinated peptide may have a sequence according to any one of SEQ ID NO: 2-55, as set forth in Table 2 below. In some embodiments, the citrullinated peptide is a cyclic citrullinated peptide (CCP).

TABLE 2
Exemplary Capture Probes

| SEQ ID NO: | Structure | Sequence |
|---|---|---|
| SEQ ID NO: 2 | Linear | QELKLXSXTKSSV |
| SEQ ID NO: 3 | Cyclic | CQELKLXSXTKSSVC |
| SEQ ID NO: 4 | Linear | EHYHYXAXGHTTT |
| SEQ ID NO: 5 | Cyclic | CEHYHYXAXGHTTTC |
| SEQ ID NO: 6 | Linear | QQFRFXGXSRAAC |
| SEQ ID NO: 7 | Cyclic | CQQFRFXGXSRAAC |
| SEQ ID NO: 8 | Linear | HEFRFXGXSRAAC |
| SEQ ID NO: 9 | Cyclic | CHEFRFXGXSRAAC |
| SEQ ID NO: 10 | Linear | HHFRFXGXSRAAC |
| SEQ ID NO: 11 | Cyclic | CHHFRFXGXSRAAC |
| SEQ ID NO: 12 | Linear | HQLRFXGXSRAAC |
| SEQ ID NO: 13 | Cyclic | CHQLRFXGXSRAAC |
| SEQ ID NO: 14 | Linear | HQFKFXGXSRAAC |
| SEQ ID NO: 15 | Cyclic | CHQFKFXGXSRAAC |
| SEQ ID NO: 16 | Linear | HQFRLXGXSRAAC |
| SEQ ID NO: 17 | Cyclic | CHQFRLXGXSRAAC |
| SEQ ID NO: 18 | Linear | HQFRFXSXSRAAC |
| SEQ ID NO: 19 | Cyclic | CHQFRFXSXSRAAC |
| SEQ ID NO: 20 | Linear | HQFRFXGXSKAAC |
| SEQ ID NO: 21 | Cyclic | CHQFRFXGXSKAAC |
| SEQ ID NO: 22 | Linear | HQFKFXGXSKAAC |
| SEQ ID NO: 23 | Cyclic | CHQFKFXGXSKAAC |
| SEQ ID NO: 24 | Linear | HQFRFXGXTRAAC |
| SEQ ID NO: 24 | Cyclic | CHQFRFXGXTRAAC |
| SEQ ID NO: 25 | Linear | HQFRFXGXSRSAC |
| SEQ ID NO: 26 | Cyclic | CHQFRFXGXSRSAC |
| SEQ ID NO: 27 | Linear | HQFRFXGXSRASC |
| SEQ ID NO: 28 | Cyclic | CHQFRFXGXSRASC |
| SEQ ID NO: 29 | Linear | HQSRFXGXSRAAC |
| SEQ ID NO: 30 | Cyclic | CHQSRFXGXSRAAC |
| SEQ ID NO: 31 | Linear | HQFRFXAXSRAAC |
| SEQ ID NO: 32 | Cyclic | CHQFRFXAXSRAAC |
| SEQ ID NO: 33 | Linear | HQFRFXGXGRAAC |
| SEQ ID NO: 34 | Cyclic | CHQFRFXGXGRAAC |
| SEQ ID NO: 35 | Linear | SHQESTRGRSRGRSGXSGS |
| SEQ ID NO: 36 | Linear | SHQESTXGXSRGRSGRSGS |
| SEQ ID NO: 37 | Linear | SHQESTXGRSXGRSGRSGS |
| SEQ ID NO: 38 | Linear | SHQESTXGRSRGRSGXSGS |
| SEQ ID NO: 39 | Linear | SHQESTXGRSRGXSGRSGS |
| SEQ ID NO: 40 | Linear | SHQESTXGRSRGRSGRSGS |
| SEQ ID NO: 41 | Linear | SHQESTRGRSXGRSGRSGS |
| SEQ ID NO: 42 | Linear | SHQESTRGRSRGXSGRSGS |
| SEQ ID NO: 43 | Linear | SHQESTRGXSRGRSGRSGS |
| SEQ ID NO: 44 | Linear | SHQESTXGRSRGRSGRSGS |
| SEQ ID NO: 45 | Linear | SHQESTXGRSRGRS |
| SEQ ID NO: 46 | Linear | SHQESTXGRSRGR |
| SEQ ID NO: 47 | Linear | SHQESTXGRSR |
| SEQ ID NO: 48 | Linear | SHQESTXGRSRG |
| SEQ ID NO: 49 | Linear | SHQESTXGRS |
| SEQ ID NO: 50 | Linear | SHQESTXGR |
| SEQ ID NO: 51 | Linear | SHQESTRGRSRGRSGXSGS |
| SEQ ID NO: 52 | Linear | SHQESTXGXSRGRSGRSGS |
| SEQ ID NO: 53 | Linear | SHQESTXGRSXGRSGRSGS |
| SEQ ID NO: 54 | Linear | SHQESTXGRSRGRSGXSGS |
| SEQ ID NO: 55 | Linear | SHQESTXGRSRGXSGRSGS |

* X = citrulline.

Thus, the invention also provides a diagnostic method of RA, which involves: (a) assaying the levels AAs; and (b) comparing the amount of AAs with a reference standard, whereby an increase or decrease in the assayed AAs compared to the standard level is indicative of a medical condition, i.e., RA.

Reference Levels. The reference level used for comparison with the measured level for an AA may vary, depending on the aspect of the invention being practiced, as will be understood from the foregoing discussion. For disease diagnostic methods, the “reference level” is typically a predetermined reference level, such as an average of levels obtained from a population that is not afflicted with RA, but in some instances, the reference level can be a mean or median level from a group of individuals including diseased patients. In some instances, the predetermined reference level is derived from (e.g., is the mean or median of) levels obtained from an age-matched population. Alternatively, the reference level may be a historical reference level for the particular patient (e.g., an AA level that was obtained from a sample derived from the same individual, but at an earlier point in time).

For disease staging or stratification methods (i.e., methods of classifying diseased patients into mild, moderate and severe stages of disease), the reference level is normally a predetermined reference level that is the mean or median of levels from a population which has been diagnosed with disease. In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

Comparing Levels of Disease-Associated AAs. The process of comparing a measured value and a reference value can be carried out in any convenient manner appropriate to the type of measured value and reference value for the disease-associated antigen or AA at issue. Measuring can be performed using quantitative or qualitative measurement techniques, and the mode of comparing a measured value and a reference value can vary depending on the measurement technology employed. For example, when a qualitative assay is used to measure disease-associated antigen or AA levels, the levels may be compared by comparing data from densitometric or spectrometric measurements (e.g., comparing numerical data or graphical data, such as bar charts, derived from the measuring device). However, it is expected that the measured values used in the methods of the invention will most commonly be quantitative values (e.g., quantitative measurements of signal intensity).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing may be manual (such as visual inspection by the practitioner of the method) or it may be automated. For example, an assay device may include circuitry and software enabling it to compare a measured value with a reference value for a disease-associated antigen or AA. Alternatively, a separate device (e.g., a digital computer) may be used to compare the measured value(s) and the reference value(s). Automated devices for comparison may include stored reference values for the disease-associated antigen or AA being measured, or they may compare the measured value(s) with reference values that are derived from contemporaneously measured reference samples.

In some embodiments, the methods of the invention utilize “simple” or “binary” comparison between the measured level(s) and the reference level(s) (e.g., the comparison between a measured level and a reference level determines whether the measured level is higher or lower than the reference level). For AA levels, a comparison showing that the measured value for the AA is higher than the reference value may indicate or suggest a diagnosis of RA. It is useful to determine appropriate partitioning of data by performing a ROC analysis. A ROC curve is a plot of the true positive rate against the false positive rate for the different possible thresholds of a diagnostic test, wherein the threshold is related to the responses of the signals from said assays. This provides a method of measuring the clinical sensitivity and specificity of a specific subset of data or the data as a whole group.

In certain aspects, the comparison is performed to determine the magnitude of the difference between the measured and reference values (e.g., comparing the “fold” or percentage difference between the measured value and the reference value). A fold difference that is about equal to or greater than the minimum fold difference disclosed herein suggests or indicates a diagnosis of a disease or medical condition, as appropriate to the particular method being practiced. A fold difference can be determined by measuring the absolute concentration of the disease-associated antigen or AA and comparing that to the absolute value of a reference, or a fold difference can be measured by the relative difference between a reference value and a sample value, where neither value is a measure of absolute concentration, and/or where both values are measured simultaneously.

As will be apparent to those of skill in the art, when replicate measurements are taken for the biomarker(s) tested, the measured value that is compared with the reference value is a value that takes into account the replicate measurements. The replicate measurements may be taken into account by using either the mean or median of the measured values as the “measured value.”

### Multiple Marker Analysis for Subject Rule-In and Rule-Out

While assays using a single capture probe are informative in the diagnosis of disease, combining the information from two or more capture probes into one diagnostic algorithm can make a substantial improvement in the prediction. By optimizing the combined information, it is possible to increase the specificity and sensitivity of the assay.

More specifically, methods of predicting whether a patient has a specific disease or stage of disease can be improved by determining the quantity of two or more autoantibodies in a sample obtained from a patient against multiple antigens such as the peptides listed in Table 2. The data collected from the two or more measurements is subjected to statistical analyses wherein the quantity of autoantibody(s) present in a sample is compared or normalized to a reference set of non-diseased samples enabling the determination of whether a specific disease is present, or alternatively, determining what stage of disease (i.e., disease progression or regression).

In a particular embodiment, the quantities obtained from the measurements are analyzed in multidimensional space (the dimensions of which comprise the responses of the signals from each of the separate assays), and the presence or absence of disease is determined by partitioning the signals on the basis of signal intensity from two or more of the measurements. It is useful to determine appropriate partitioning of data by performing a ROC analysis. A ROC curve is a plot of the true positive rate against the false positive rate for the different possible thresholds of a diagnostic test, wherein the threshold is related to the responses of the signals from said assays. This provides a method of measuring the clinical sensitivity and specificity of a specific subset of data or the data as a whole group. The two or more measurements may consist of measuring variants of autoantibodies present in a sample with different capture agents (e.g., different antigens such as those listed in Table 2) and/or different x-human Ig antibodies (e.g., x-IgM versus x-IgG antibodies). The difference between x-IgM and x-IgG antibodies may provide information regarding the stage of disease.

### Prognostic or Predictive Assays

The disclosure also provides for prognostic (or predictive) assays for determining whether an individual is at risk of developing a condition, disorder or disease associated with the presence or absence of AAs. Such assays can be used for prognostic or predictive purpose, for example to thereby prophylactically treat an individual prior to the onset of a disorder characterized by or associated with AAs, e.g., rheumatoid arthritis. The methods described herein can also be used to determine the levels of such AAs in subjects to aid in predicting the response of such subjects to medication. Another aspect of the invention provides methods for determining an AA expression in an individual to thereby select appropriate therapeutic or prophylactic compounds for that individual.

Accordingly, the prognostic assays described herein can be used to determine whether a subject can be administered a compound (e.g., an agonist, antagonist, peptidomimetic, polypeptide, peptide, nucleic acid, small molecule, or other drug candidate) to treat a disease or condition associated with the presence of AAs. Thus, the invention provides methods for determining whether a subject can be effectively treated with a compound for a disorder or condition associated with an aberrant AA levels or in which a test sample is obtained and the AAs are detected using the assays described herein (e.g., wherein the presence, absence, and/or amount of the AAs is diagnostic for a subject that can be administered the compound to treat a disorder associated with an aberrant AA level).

The level of the AAs in a sample obtained from a subject is determined and compared with the level found in a obtained from a different subject (or population of subjects) who is free of the condition, in an earlier or later stage of the condition, has a more or less severe form of the condition or responds differently to treatments of the condition. An overabundance (or under abundance) of the AAs in the sample obtained from the subject suspected of having the condition affecting AA levels compared with the sample obtained from the different subject or population is indicative of the condition in the subject being tested.

The methods described herein can be performed, e.g., by utilizing pre-packaged diagnostic kits comprising at least one probe reagent, which can be conveniently used, e.g., in clinical settings diagnosis or prognosis subjects exhibiting symptoms of the condition.

Correlating a Subject to a Standard Reference Population. To deduce a correlation between clinical response to a treatment and a particular level of AAs, it is necessary to obtain data on the clinical responses exhibited by a population of individuals who received the treatment, i.e., a clinical population. This clinical data maybe obtained by retrospective analysis of the results of a clinical trial(s). Alternatively, the clinical data may be obtained by designing and carrying out one or more new clinical trials. The analysis of clinical population data is useful to define a standard reference population(s) which, in turn, are useful to classify subjects for clinical trial enrollment or for selection of therapeutic treatment. In one embodiment, the subjects included in the clinical population have been graded for the existence of the medical condition of interest. Grading of potential subjects can include, e.g., a standard physical exam or one or more lab tests. Alternatively, grading of subjects can include use of a biomarker expression pattern. For example, AA level is a useful as grading criteria where there is a strong correlation between expression pattern and susceptibility or severity to a disease or condition. In one embodiment, a subject is classified or assigned to a particular group or class based on similarity between the measured levels of AA in the subject and the level of the AA observed in a standard reference population.

In one embodiment, a treatment of interest is administered to each subject in a trial population, and each subject's response to the treatment is measured using one or more predetermined criteria. It is contemplated that in many cases, the trial population will exhibit a range of responses, and that the investigator will choose the number of responder groups (e.g., low, medium, high) made up by the various responses. In addition, the expression level of a biomarker (e.g., AAs) is quantified, which may be done before and/or after administering the treatment. These results are then analyzed to determine if any observed variation in clinical response between groups is statistically significant. Statistical analysis methods, which may be used, are described in L. D. Fisher & G. vanBelle, Biostatistics: A Methodology for the Health Sciences (Wiley-Interscience, New York (1993)).

The skilled artisan can construct a mathematical model that predicts clinical response as a function of the level of AAs from the analyses described above. The identification of an association between a clinical response and an expression level for the AAs may be the basis for designing a diagnostic method to determine those individuals who will or will not respond to the treatment, or alternatively, will respond at a lower level and thus may require more treatment, i.e., a greater dose of a drug. The only requirement is that there be a good correlation between the diagnostic test results and the underlying condition. In one embodiment, this diagnostic method uses an assay for AAs described above.

Monitoring Clinical Efficacy. In one embodiment, the present invention provides for monitoring the influence of treatments (e.g., drugs, compounds, small molecules or devices) on the level of AAs. Such assays can also be applied in basic drug screening and in clinical trials. For example, the effectiveness of an agent to increase (or decrease) autoantibody levels can be monitored in clinical trials of subjects. An agent that affects the level of AAs can be identified by administering the agent and observing a response. In this way, the level of the AAs can serve as a marker, indicative of the physiological response of the subject to the agent. Accordingly, this response state may be determined before, and at various points during, treatment of the individual with the agent.

Subject Classification. Standard control levels of AAs are determined by measuring levels in different control groups. The control levels are then compared with the measured level of AAs in a given subject. The subject can be classified or assigned to a particular group based on how similar the measured levels were compared to the control levels for a given group.

As one of skill in the art will understand, there will be a certain degree of uncertainty involved in making this determination. Therefore, the standard deviations of the control group levels can be used to make a probabilistic determination and the method of this invention are applicable over a wide range of probability-based group determinations. Thus, for example, and not by way of limitation, in one embodiment, if the measured level of the AAs falls within 2.5 standard deviations of the mean of any of the control groups, then that individual may be assigned to that group. In another embodiment, if the measured level of the AAs falls within 2.0 standard deviations of the mean of any of the control groups then that individual may be assigned to that group. In still another embodiment, if the measured level of the AAs fall within 1.5 standard deviations of the mean of any of the control groups then that individual may be assigned to that group. In yet another embodiment, if the measured level of the AAs is 1.0 or less standard deviations of the mean of any of the control groups levels then that individual may be assigned to that group. Thus, this process allows determination, with various degrees of probability, which group a specific subject should be placed in, and such assignment would then determine the risk category into which the individual should be placed.

### Substrates

In some embodiments, capture probes may be immobilized on a substrate, i.e., solid support. Examples of such solid supports include plastics such as polycarbonate, complex carbohydrates such as agarose and sepharose, acrylic resins and such as polyacrylamide and latex beads, magnetic beads, and glass slides or glass slides functionalized for attachment of biomolecules. Other examples include SurModic Codelink or Schott Hydrogel slides. Techniques for coupling biomolecules to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

Appropriate linkers, which can be cross-linking agents, for conjugating a ligand to a solid support include a variety of agents that can react with a functional group present on a surface of the support, or with the ligand, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

A capture probe, such as a polypeptide can be immobilized on a solid support, such as a coated slide, through a covalent amide bond formed between a carboxyl group functionalized substrate and the amino terminus of the polypeptide or, conversely, through a covalent amide bond formed between an amino group functionalized substrate and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a patch at the bottom of a well of a solid support or on the flat surface of a solid support.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

A captive probe can be conjugated to a solid support through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide containing a complementary binding moiety. For example, a bead coated with avidin or with streptavidin can be bound to a polypeptide having a biotin moiety incorporated therein, or to a second solid support coated with biotin or derivative of biotin, such as imino-biotin. Additionally, a peptide can be covalently conjugated to another carrier protein. The carrier protein could be, for example, Bovine Serum Albumin (BSA), where the coupling takes place using covalent or non-covalent conjugation of the peptide and the carrier protein. The resulting conjugate can be immobilized on a solid support. Alternatively, the carrier protein (e.g. streptavidin or BSA) can be immobilized to a substrate first, followed by immobilization of the peptide.

It should be recognized that any of the binding agents disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

Any suitable substrate may be used and such substrates may be addressable. A plurality of capture probes (e.g. antigens or antigens coupled to a carrier molecule), each of which can recognize a different target analyte (e.g. autoantibodies), may be attached to the substrate in an array of spots. If desired, each spot of capture probes may be located between two electrodes, the optional label on the detection probe may be a nanoparticle made of a material that is a conductor of electricity, and a change in conductivity may be detected. For example, the electrodes may be made of gold and nanoparticles may be made of gold.

In some embodiments, the methods described herein may detect disease-associated AAs through a specific binding of a nanoparticle-based detection probe with the autoantibody. The signal from the nanoparticles may be amplified with a silver or gold enhancement solution from any substrate which allows observation of the detectable change. Suitable substrates include transparent or opaque solid surfaces (e.g., glass, quartz, plastics and other polymers TLC silica plates, filter paper, glass fiber filters, cellulose nitrate membranes, nylon membranes), and conducting solid surfaces (e.g., indium-tin-oxide (ITO, silicon dioxide (SiO₂), silicon oxide (SiO), silicon nitride, etc.)). The substrate can be any shape or thickness, but generally will be flat and thin like a microscope slide or shaped into well chambers like a microtiter plate.

### Detection Probes

In some embodiments, the capture probes bound to the solid support specifically bind to a corresponding molecule to form a complex. Simultaneously or subsequently, the molecule is contacted with a detection probe. In one embodiment, the detection probes are coupled with a label moiety, i.e., detectable group. The particular label or detectable group conjugated to the binding agent is not a critical aspect of the invention, so long as it does not significantly interfere with the specific binding of the binding agent to the target molecule, i.e., human immunoglobulin. In a particular embodiment, the detection probe comprises a nanoparticle conjugated directly or indirectly to an anti-human Ig antibody, e.g., one or more of an anti-IgG (including AAs that possess Fc domains), anti-IgA, anti-IgM, anti-IgE, and anti-IgD. The nanoparticle-antibody conjugate is contacted with the substrate under conditions effective to allow binding of the target molecule (e.g., AAs) on the substrate with the anti-human Ig antibody.

Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂ Se₃, Cd₃ P₂, Cd₃ As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods. Other nanoparticles useful in the invention include silica and polymer (e.g., latex) nanoparticles.

Previous studies have demonstrated that biomolecules including DNA and antibodies can be conjugated to gold nanoparticles via a thiol linkage (Mirkin et al., Nature 382:607-609 (1996)). The resulting modified gold particles can be used to detect analytes in a variety of formats (See, e.g., Storhoff et al., Chem. Rev., 99:1849-1862 (1999); Niemeyer, C. M. Angew. Chem. Int. Ed., 40:4128-4158 (2001); Liu et al., J. Am. Chem. Soc., 125:6642-6643 (2003)), including DNA microarrays, where high detection sensitivity is achieved in conjunction with silver amplification (Taton et al., Science, 289:1757-1760 (2000); Storhoff et al., Biosens. Bioelectron, 19:875-883 (2004)).

An effective method for functionalizing nanoparticles with biomolecules has been developed. See U.S. Pat. Nos. 6,361,944 and 6,417,340 (Nanosphere, Inc.), which are incorporated by reference in their entirety. The process leads to nanoparticles that are heavily functionalized and have enhanced particle stability. The resulting modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Such methods can also be used to generate nanoparticle-antibody or nanoparticle-biotin conjugates.

In other embodiments, the detectable group can be any material having a detectable physical or chemical property. Such detectable labels have been well-developed in the field of immunoassays and imaging, in general, most any label useful in such methods can be applied to the present invention. Useful labels include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹³¹I, ¹¹²In, ^{99m}Tc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6th Ed., Molecular Probes, Inc., Eugene Oreg.).

The nanoparticle may be linked to an antibody either directly or indirectly. For example, the nanoparticle may be directly functionalized with the desired detection antibody. Alternatively, the nanoparticle may be functionalized with a biotin moiety and the desired detection antibody is also functionalized with a biotin moiety. An avidin or streptavidin molecule is used to link (i.e., “bridge”) the nanoparticle to the antibody. The antibody-nanoparticle conjugate may be formed by step-wise addition of the antibody, streptavidin, and biotinylated nanoparticle to the substrate. For example, see U.S. Provisional Application Ser. No. 61/036,892 filed on Mar. 14, 2008, which is hereby incorporated by reference herein in its entirety and U.S. Provisional Application Ser. No. 61/055,875 filed on May 23, 2008, which is hereby incorporated by reference herein in its entirety. Receptor-ligand pairs alternative to streptavidin-biotin also may be used. For instance, the FITC anti-FITC system is a well known alternative to biotin streptavidin. Additionally, double-headed protease inhibitors (Black-eyed pea chymotrypsin or trypsin inhibitor) bind two molecules of protease simultaneously (Gennis et al., J. Biol. Chem., 251:741-746). As such, the inhibitors can be used to link the nanoparticle and the antibody using two connecting genetically modified proteases.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labelling moieties, include, but are not limited to, e.g., fluorescein and its derivatives, rhodamine and its derivatives, dansyl, umbelliferone, and the like. Chemiluminescent compounds useful as labelling moieties, include, but are not limited to, e.g., luciferin, and 2,3-dihydrophthalazinediones, e.g., luminol. For a review of various labeling or signal-producing systems which can be used, see, U.S. Pat. No. 4,391,904.

### Detection

Means of detecting labels are well known to those of skill in the art. Thus, for example, where the label is a radioactive label, means for detection include a scintillation counter or photographic film for autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label.

In some embodiments, a colorimetric method for monitoring scattered light may be used to detect the nanoparticle conjugates. See U.S. Ser. No. 10/995,051, filed Nov. 22, 2004, which is incorporated by reference in its entirety. Moreover, the methods enable the detection of probe-target complexes containing two or more particles in the presence of a significant excess of non-complexed particles, which drives hybridization in the presence of low target concentrations.

Nanoparticle detection probes, particularly gold nanoparticle probes conjugated to antibodies, are suitable for detection of AAs. A silver-based signal amplification procedure can further provide ultra-high sensitivity enhancement. Silver staining can be employed with any type of nanoparticles that catalyze the reduction of silver and can be used to produce or enhance a detectable change in any assay performed on a substrate, including those described above.

A nanoparticle can also be detected, for example, using resonance light scattering, after illumination by various methods including dark-field microscopy, evanescent waveguides, or planar illumination of glass substrates. Metal particles>40 nm diameter scatter light of a specific color at the surface plasmon resonance frequency (Yguerabide et al., Anal. Biochem., 262:157-176 (1998)), and can be used for multicolor labeling on substrates by controlling particle size, shape, and chemical composition (Taton et al., J. Am. Chem. Soc., 123:5164-5165 (2001); Jin et al., Science, 294:1901-1903 (2001)). In another embodiment, a nanoparticle can be detected in a method of the invention, for example, using surface enhanced raman spectroscopy (SERS) in either a homogeneous solution based on nanoparticle aggregation (Graham et al., Angew. Chem., 112:1103 (2000)), or on substrates in a solid-phase assay (Porter et al., Anal. Chem., 71:4903-4908 (1999)), or using silver development followed by SERS (Mirkin et al., Science, 297:1536-1540 (2002)). In another embodiment, the nanoparticles may be detected by photothermal imaging (Boyer et al., Science, 297:1160-1163 (2002)), diffraction-based sensing technology (Bailey et. al, J. Am Chem. Soc., 125:13541 (2003)), or hyper-Rayleigh scattering (Kim et al., Chem Phys. Lett., 352:421 (2002)).

A nanoparticle can be detected in a method of the invention, for example, using an optical or flatbed scanner. The scanner can be linked to a computer loaded with software capable of calculating grayscale measurements, and the grayscale measurements are calculated to provide a quantitative measure of the amount of analyte detected. Suitable scanners include those used to scan documents into a computer which are capable of operating in the reflective mode (e.g., a flatbed scanner), other devices capable of performing this function or which utilize the same type of optics, any type of grayscale-sensitive measurement device, and standard scanners which have been modified to scan substrates according to the invention. The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

### Kits

Also within the scope of the disclosure are kits comprising capture and detection probe compositions and instructions for use. The kits are useful for detecting the presence of AAs in a biological sample, e.g., any body fluid including, but not limited to, serum, plasma, lymph, cystic fluid, urine, stool, cerebrospinal fluid, acitic fluid or blood and including biopsy samples of body tissue. For example, the kit can comprise: one or more capture probes and/or detection probes; means for determining the amount of the AAs in the sample; and means for comparing the amount of the AAs in the sample with a standard. One or more of the detection probes may be labeled. The kit components, (e.g., reagents) can be packaged in a suitable container. The kit can further comprise instructions for using the kit to detect the AAs.

In one embodiment, the kit includes: (1) a capture probe (e.g., antigen for disease-associated AA); and (2) an antibody which binds to the AAs and is conjugated (directly or indirectly) to a nanoparticle. The kit can also include, e.g., a buffering agent, a preservative or a protein-stabilizing agent. The kit can further include components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits may contain a written product on or in the kit container. The written product describes how to use the reagents contained in the kit, e.g., to use the AAs in determining a strategy for preventing or treating RA in a subject. In several embodiments, the use of the reagents can be according to the methods described herein.

### EXAMPLES

The present invention is further illustrated by the following examples, which should not be construed as limiting in any way.

The representative Examples below demonstrate the efficacy and utility of the disclosed methods for detecting AAs using modified gold nanoparticles. Previous studies have demonstrated that biomolecules including DNA and antibodies can be conjugated to gold nanoparticles via a thiol linkage (Mirkin et al., Nature 382:607-609 (1996)). The resulting modified gold particles have been used to detect analytes in a variety of formats (See, e.g., Storhoff et al., Chem. Rev., 99:1849-1862 (1999); Niemeyer, C. M. Angew. Chem. Int. Ed., 40:4128-4158 (2001); Liu et al., J. Am. Chem. Soc., 125:6642-6643 (2003)), including DNA microarrays, where high detection sensitivity is achieved in conjunction with silver amplification (Taton et al., Science, 289:1757-1760 (2000); Storhoff et al., Biosens. Bioelectron, 19:875-883 (2004)). Additional key features of this technology include the remarkable stability and robustness of the modified gold nanoparticles which withstand both elevated temperatures and salt concentrations (Mirkin et al. Nature, 382:607-609 (1996); Storhoff et al., Langmuir, 18:6666-6670 (2002)), as well as the remarkable specificity by which target analytes are recognized (Storhoff et al., J. Am. Chem. Soc., 120:1959-1964 (1998); Taton et al., Am. Chem. Soc., 122:6305-6306 (2000)).

### Example 1

### Preparation of Gold Nanoparticles

Gold colloids (about 15 nm diameter) are prepared by reduction of HAuCl₄ with citrate as described in Frens, Nature Phys. Sci., 241:20-22 (1973) and Grabar, Anal. Chem., 67:735 (1995). Briefly, all glassware is cleaned in aqua regia (3 parts HCl, 1 part HNO₃), rinsed with Nanopure H₂O, then oven dried prior to use. HAuCl₄ and sodium citrate are purchased from Aldrich Chemical Company. Aqueous HAuCl₄ (1 mM, 500 mL) is brought to reflux while stirring. Then, 38.8 mM sodium citrate (50 mL) is added quickly. The solution color changed from pale yellow to burgundy, and refluxing is continued for 15 min. After cooling to room temperature, the red solution is filtered through a Micron Separations Inc. 0.2 micron cellulose acetate filter. Au colloids are characterized by UV-vis spectroscopy using a Hewlett Packard 8452A diode array spectrophotometer and by Transmission Electron Microscopy (TEM) using a Hitachi 8100 transmission electron microscope.

### Example 2

### Preparation of Antigen-Coated Substrates

Purified capture probe (e.g., any one or more of the antigens recognized by AAs in RA subjects described above) are synthesized according to standard procedures. The proteins or peptides are arrayed onto Codelink (Amersham, Inc.) or Hydrogel substrates (Nexterion Slide H Hydrogel Coated Substrate) using a GMS417 arrayer (Affymetrix). The substrates are incubated overnight in a humidity chamber, and subsequently washed with TBS-T Buffer (150 mM NaCl/10 mM Tris Base buffer (pH 8) containing 0.05% Tween. All of the proteins are arrayed in triplicate. The position of the arrayed spots is designed to allow multiple assays on each substrate, achieved by partitioning the substrate into separate test wells by silicon gaskets (Grace Biolabs). For example, the following capture probes are arrayed on a slide:

TABLE 3
Preparation of Illustrative Arrays of Capture Probes

| Sample | Capture Probe | Water (μL) | 4X Printing Buffer (μL) |
|---|---|---|---|
| 1 | 100 μL of BSA | 50 | 50 |
| | (80 ng/μL) | | |
| 2 | 50 μL of BSA | 100 | 50 |
| | (80 ng/μL) | | |
| 3 | 25 μL of BSA | 125 | 50 |
| | (80 ng/μL) | | |
| 4 | 5 μL of BSA | 145 | 50 |
| | (80 ng/μL) | | |
| 5 | 30 μL peptide | 60 | 30 |
| | antigen (80 ng/μL) | | |
| 6 | 30 μL peptide | 87 | 3 |
| | antigen (80 ng/μL) | | |
| 7 | 60 μL peptide | 30 | 30 |
| | antigen (80 ng/μL) | | |
| 8 | 60 μL peptide | 57 | 3 |
| | antigen (80 ng/μL) | | |
| 9 | 85 μL of Filaggrin | 74 | 53 |
| | (100 ng/μL) | | |
| 10 | 106 μL sample 9 | | 106 |
| 11 | 106 μL sample 10 | | 106 |
| 12 | 40 μL sample 11 | | 160 |

Following binding, the slides are rinsed two times with 1×PBS/0.3% Tween (200 μL). The slides are then incubated with blocking solution (25 mM NaCl/25 mM Tris, pH 8.0/25 mM ethanolamine/0.15% Tween 20/0.5×PBS/0.5% BSA) for Codelink and Hydrogel slides at room temperature (23° C.), 250 rpm for 60 min. Finally, the slides are rinsed two times with 150 mM NaNO₃/0.3% Tween.

### Example 3

### Detection of AAs

In an illustrative embodiment, test samples are assayed using the array prepared as described in Example 2 above. The assay conditions are as follows. One hundred microliters (100 μL) of the samples (1% serum sample dilution) are added to each well and incubated at room temperature, with shaking at 250 rpm for 10 min. Next, the target binding solution is shaken off and the plate is washed three times with 150 mM NaNO₃/0.3% TW. A biotin-antibody mixture (100 μL of 50 ng/100 μL in binding buffer) is added to each well and the slides are incubated at 23° C., with 250 rpm shaking for 10 min. The biotin antibody mixture comprises IgA+IgG+IgM (KPL, Cat# 16-10-07). The target binding solution is removed and the plates are washed three times with 150 mM NaNO₃/0.3% Tween. Next, free streptavidin (SA) (10 ng/μL) is allowed to bind by adding 100 μL to each well. The slides are incubated at 23° C., with 250 rpm shaking for 10 min. The SA solution is removed and the plates are washed three times with 150 mM NaNO₃/0.3% TW. Next, 100 μL of Biotin-conjugated gold nanoparticle probe (0.214 μL biotin-Au probe/100 uL binding buffer) is added each well and the slides are incubated at 23° C. with shaking at 250 rpm for 10 min. The nanoparticle solution is removed and the plates are washed two times with 150 mM NaNO₃/0.3% Tween.

Silver development is then used to enhance the images. Briefly, silver solutions A (Part # E700074D007) and B6 (Part # E700251D001) are mixed in a 50 mL of tube and added to a slide container. The slides are incubated at 120 rpm for 5.5 min at room temperature (23° C.). After silver development, the slides are rinsed with copious amounts of deionized water (at least 100 mL/slide). The slides are dried by spinning and the back of the slides are cleaned with a soft cloth or tissue. Finally, the slides are imaged with the Verigene System at 1.8 ms, 3.9 ms and multiple exposures 6×(10 ms, 20 ms, 50 ms, 100 ms, 200 ms, 500 ms, 1000 ms). Signal is the relative numerical signal response taken from the image of a scan from a Tecan LS scanner with data extraction and quantitation performed using GenePix software (Axon Instruments).

### EQUIVALENTS

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 units refers to groups having 1, 2, or 3 units. Similarly, a group having 1-5 units refers to groups having 1, 2, 3, 4, or 5 units, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

All publications, patent applications, patents, and other references mentioned herein are expressly incorporated by reference in their entirety, to the same extent as if each were incorporated by reference individually. In case of conflict, the present specification, including definitions, will control.

1. An antigen consisting of a linear or cyclic peptide according to any of SEQ ID NO: 2-56 that is specifically immunoreactive with autoantibodies present in the serum of subjects with autoimmune disease.

2. The antigen of claim 1, wherein the autoantibodies are anti-filaggrin antibodies present in the serum of subjects suffering from rheumatoid arthritis

3. A method for the diagnosis of rheumatoid arthritis in a subject comprising:
(a) providing a substrate having a capture probe bound thereto, wherein the capture probe comprises an antigen recognized by autoantibodies present in the serum of subjects suffering from rheumatoid arthritis;
(b) contacting the substrate having the capture probe bound thereto with (i) a sample from the subject and (ii) a detection probe under conditions that are suitable for the formation of a complex of the capture probe and detection probe with the autoantibodies, if present in the sample, wherein the detection probe comprises a nanoparticle and a binding agent that specifically binds to the autoantibodies; and
(c) detecting the formation of the complex of the capture probe and detection probe with the autoantibodies, wherein the presence of the complex is indicative of rheumatoid arthritis in the subject.

4. The method of claim 3, wherein the autoantibodies present in the serum of subjects suffering from rheumatoid arthritis are antifilaggrin autoantibodies or epitope-related autoantibodies.

5. The method of claim 3, wherein the antigen recognized by autoantibodies present in the serum of subjects suffering from rheumatoid arthritis is a citrullinated peptide.

6. The method of claim 5, wherein the citrullinated peptide is a cyclic citrullinated peptide.

7. The method of claim 5, wherein the citrullinated peptide has a sequence according to any of SEQ ID NOs: 2-56.

8. The method of claim 3, wherein the sample is first contacted with the detection probe and then contacted with the capture probe.

9. The method of claim 3, wherein the sample is first contacted with the capture probe and then contacted with the detection probe.

10. The method of claim 3, wherein the sample, the detection probe, and the capture probe are contacted simultaneously.

11. The method of claim 3, wherein the detection probe further comprises a fluorophore, a phosphor, a quantum dot, an enzyme conjugate, or a avidin/biotin conjugate.

12. The method of claim 13 wherein the binding agent that specifically binds to the autoantibodies is an anti-human Ig antibody.

13. The method of claim 12, wherein the anti-human antibody is selected from the group consisting of: anti-human IgG, anti-human IgM, anti-human IgA, anti-human IgE, anti-human IgD, and subtypes or mixtures thereof.

14. The method of claim 3, wherein the nanoparticle is conjugated directly to the binding agent.

15. The method of claim 3, wherein the nanoparticle is conjugated indirectly to the binding agent by a bridge or linker molecule.

16. The method of claim 15, wherein the nanoparticle and binding agent are each conjugated to biotin and the nanoparticle and second binding agent are joined by an avidin or streptavidin bridge.

17. The method of claim 3, wherein the complex is detected by photonic, electronic, acoustic, opto-acoustic, gravitic, electro-chemical, electro-optic, mass-spectrometric, enzymatic, chemical, biochemical, magnetic, paramagnetic, or physical means.

18. The method of claim 3, wherein the nanoparticles are made of a noble metal.

19. The method of claim 18, wherein the nanoparticles are made of gold or silver.

20. The method of claim 3, wherein the substrate is a nanoparticle, a thin film, or a magnetic bead.

21. The method of claim 3, wherein the substrate has a planar surface.

22. The method of claim 3, wherein the substrate is made of glass, quartz, ceramic, or plastic.

23. The method of claim 3, wherein the detecting comprises contacting the substrate with silver stain.

24. The method of claim 3, wherein the detecting comprises detecting light scattered by the nanoparticles.

25. The method of claim 3, wherein the substrate is addressable.

26. The method of claim 3, wherein the sample is blood, plasma, or serum.
